(19) United States
(12) Patent Application Publication
(10) Pub. No.: US 2006/0088842 A1
(43) Pub. Date: Apr. 27, 2006

(54) RT-PCR-based cloning of human SMN, the SMA determining gene, and the construction of its expression plasmids

(76) Inventor: Khue Vu Nguyen, San Diego, CA (US)

Correspondence Address: H-Mai T. Nguyen, Ph.D., 2120-C Las Palmas Drive, Carlsbad, CA, 92009, US

(21) Appl. No.: 10/777,691

(22) Filed: Feb. 13, 2004

(51) Int. Cl. C12Q 1/68; C12P 19/34; C12N 15/87

(52) U.S. Cl. 435/6; 435/91.2; 435/455

(57) ABSTRACT

Spinal muscular atrophy (SMA) is a lethal autosomal recessive disease. The gene most highly associated with SMA is the survival motor neuron (SMN) gene. The present invention is a new procedure for the cloning of human SMN, the SMA determining gene, based on the reverse transcription (RT) and the polymerase chain reaction (PCR). This procedure for cloning human SMN gene by means of RT-PCR reactions is cost-effective, not time-consuming, and is suited for any laboratory. The present invention also includes a new procedure for the construction of expression plasmids, a/ using the pFastBac™ HTb and the pBlueBacHis2 A transfer vectors for the purpose of obtaining human SMN protein in insect cells; and b/ using the pET-28a (+) transfer vector for the purpose of obtaining human SMN protein in bacteria. The present invention makes it easier to obtain full-length SMN protein which is valuable for biochemical and biological analyses that may elucidate the molecular mechanism of SMA. Knowing the molecular mechanism of SMA will allow the exploration of gene therapy in SMA.

### I. FIELD OF THE INVENTION

Spinal muscular atrophy (SMA) is a lethal autosomal recessive disease. SMA is characterized by the degeneration of motor neurons in the spinal cord, causing progressive weakness of the limbs and trunk, followed by muscle atrophy. The gene most highly associated with SMA is the survival motor neuron (SMN) gene. The availability of full-length human SMN protein will be valuable for biochemical and biological analyses that may elucidate the molecular mechanism of SMA. The present invention is a new approach to the cloning of human SMN gene based on the reverse transcription (RT) and the polymerase chain reaction (PCR), and the construction of expression plasmids in order to obtain full-length human SMN protein.

### II. BACKGROUND OF THE INVENTION

Spinal muscular atrophy (SMA) is characterized by the degeneration of anterior horn cells of the spinal cord, leading to progressive symmetrical limb and trunk paralysis and muscular atrophy. It is the second most common fatal autosomal recessive disorder after cystic fibrosis and is the common genetic cause of childhood mortality (1-4). Childhood spinal muscular atrophies are divided into three clinical groups on the basis of age of onset and clinical course (5). The so-called Werdnig-Hoffman disease (6,7), or SMA type I (SMA-I), is characterized by severe, generalized muscle weakness and hypotonia that is noticeable at birth or appears within the first 3 months of life. In this acute form of the disease, death by respiratory failure usually occurs within the first 2 years. Type I SMA patients are never able to sit or walk. Children who have the intermediate form, SMA type II (SMA-II), can sit but cannot stand or walk unaided. They survive beyond 4 years of age until adolescence or later. Patients with Kugelberg-Welander disease (8), or SMA type III (SMA-1H), display proximal muscle weakness that begins to develop after 2 years of age and able to stand and walk, but often become wheelchair-bound during youth or adulthood.

All three types of SMA map to chromosome 5q 13.3, suggesting that they are the result of allelic mutations (9-12). The genomic region encompassing the disease gene is particularly unstable and prone to large scale deletions (13). The molecular mechanism of motor neuron death in SMA remains unknown, but two genes associated with SMA have been identified in the following region: Survival motor neuron, SMN (14) and neuronal apoptosis inhibitory protein (NAIP) (15). Lefebvre et al. (14) identified the SMN gene with 8 exons extending over approximately 20 kb. It was found that the SMN gene was either absent or interrupted in its exons 7 and 8 in the majority of patients (98%), independently of the type of SMA, suggesting that SMN is the determining gene (14). Roy et al. (15) identified the NAIP gene with 16 exons spanning 60 kb. It was shown that 45% of SMA type 1 and 18% of SMA type II and III patients have a partial or complete homozygous deletions of the NAIP gene. Since the NAIP gene is very close to the SMN gene, it is hypothesized that the mutations in the SMN gene are the major determinant of the SMA phenotype, whereas the extent of the deletions, which may include the NAIP gene, may modify the SMA phenotype thus accounting for the different clinical subtypes of the disease (14, 15). The SMN gene encodes a 38-kDa protein ubiquitously expressed, the SMN protein, which reveals no significant sequence homology with other protein. Its exact function is actually unknown, but it interacts with several other proteins involved in spliceosomal assembly, suggesting a role for SMN in pre-mRNA splicing (16-18). SMN is an essential protein since a knockout mouse for the SMN gene is lethal embryonically (19). A study of the distribution of the SMN protein complex in human fetal tissues showed interesting results in favor of the “muscle hypothesis” of SMA (20). In fetal muscle cells, SMN protein was concentrated in large cytoplasmic dot-like structures similar to the size of a gem (the nuclear structure containing the SMN protein), which were never previously described in the cytoplasm. This immunolocalization of SMN protein in skeletal muscle contrasts with results obtained in other normal tissues, such as thymus, kidney, lung, and brain or liver and spinal cord (21). Animal models for SMA are recent (22-24). Thus, the availability of full-length SMN protein is valuable for biochemical and biological analyses that may elucidate the molecular mechanism of SMA. Currently, the procedure to obtain full-length SMN protein is the isolation of SMN-cDNA clones from the cDNA library using the synthesized oligonucleotide probes; this method of probing to identify appropriate SMN-cDNA for cloning is costly, very time-consuming (requiring labor intensive steps), and requires highly skilled personnel. Consequently, very few laboratories have the equipment or capability to produce full-length SMN protein; for that reason, other laboratories, which do not have such capability, would have to obtain SMN-cDNA clones from the few laboratories that can produce SMN-cDNA clones. Therefore, there is a need to design an easy, simple and cost-effective procedure to obtain SMN-cDNA for cloning. For such a purpose, in the present invention, a different approach is used: Instead of using probes to isolate SMN-cDNA clones, the new procedure involves starting with the SMN-messenger ribonucleic acid (SMN-mRNA) and utilizing the RT and PCR reactions in order to obtain SMN-cDNA for cloning. This is a simple and cost-effective procedure that can be performed in any laboratory.

In addition, the cloning of SMN-cDNA based on RT-PCR reactions allows the construction of expression plasmids, (a) using the pFastBac™ HTb and the pBlueBacHis2 A transfer vectors for the purpose of obtaining human SMN protein in insect cells; and (b) using the pET-28a (+) transfer vector for the purpose of obtaining human SMN protein in bacteria.

### III. PURPOSE OF THE INVENTION

The purpose of the invention is: 1/ To perform the procedure to obtain SMN-cDNA for cloning by starting with SMN-messenger ribonucleic acid (SMN-mRNA) and by utilizing the RT and PCR reactions; and 2/ to construct expression plasmids (a) using the pFastBac™ HTb and the pBlueBacHis2 A transfer vectors for the purpose of obtaining human SMN protein in insect cells; (b) using the pET-28a (+) transfer vector for the purpose of obtaining human SMN protein in bacteria. The availability of full-length SMN protein is essential for the identification of biochemical and biological activities that occur in the SMN protein which will allow the exploration of gene therapy in SMA.

### IV. MATERIALS AND METHODS

Isolation of RNA

The total ribonucleic acid (RNA) was isolated from a sample of human liver biopsy (wild type) according to the method described by Sambrook et al. (25) using guanidin/phenol (Tris Reagent™, Euromedex, 67460 Souffelweyersheim, France). The total RNA was dissolved in water pre-treated by 0.1% diethyl pyrocarbonate (DEPC, Sigma, St. Louis, Mo.). This RNA solution is ready for subsequent treatment for the synthesis of the cDNA.

Reverse Transcription

The synthesis of the cDNA was performed by reverse transcription (RT), as described by Sambrook et al. (25). The first copies of cDNA were synthesized by using the synthesized oligonucleotide (SEQ ID NO. 1) (Genosys Biotechnologies, Europe, Ltd., France) with the following sequence: 5′ TGGCAGACTTAC 3′ (SEQ ID NO. 1). This oligonucleotide was selected by taking the complementary sequence between base pairs 889 and 900 of the human mRNA for SMN described by Lefebvre et al. (14). The M-MLV Reverse Transcriptase enzyme (Gibco BRL®, Life Technologies Sarl, BP 96, 95613 Cergy Pontoise, France) was used in order to perform the reverse transcription reaction. This reaction was performed as follow:

0.1 nmol of oligonucleotide SEQ ID NO. 1, and 10 pmol each of nucleotides dATP, dCTP, dGTP and dTTP were added to 5 μg of the total RNA. The reaction was conducted in the presence of a reaction buffer for RT of the Gibco BRL® kit; the total volume of the reaction was 20 μl. After heating the mixture to 90° C. for 2 minutes and then cooling it on ice for 1 minute, 200 U M-MLV were added; then the mixture was left at 25° C. for 10 minutes and then at 42° C. for 45 minutes.

Amplification

Having obtained the first copies of cDNA by RT reaction, the next step is to amplify the RT products.

Amplifying the RT products were assessed by using the polymerase chain reaction (PCR) technique (26, 27). Two synthesized oligonucleotides (SEQ ID NO. 2) (forward primer) and (SEQ ID NO. 3) (reverse primer) (Genosys Biotechnologies) were used in order to perform the PCR reaction. They have the following sequences:

| | |
|---|---|
| 5′ ATGGCGATGAGCAGCGG 3′ | (SEQ ID NO.2) |
| and | |
| 5′ TTAATTTAAGGAATGTGAGCAC 3′ | (SEQ ID NO.3) |

The oligonucleotide SEQ ID NO. 2 was based on the sequence between base pairs 1 and 17 of the human mRNA sequence for SMN described by Lefebvre et al. (14). The oligonucleotide SEQ ID NO. 3 was selected by taking the complementary sequence between base pairs 864 and 885 of the human mRNA sequence for SMN described by Lefebvre et al. (14). Amplification was conducted by using a DNA Thermal Cycle (Amplitron® II Thermolyse). The reaction was conducted in a total volume of 50 μl with 2.5 U of Taq DNA polymerase (Promega Corporation, Madison, Wis., U.S.A.) in the presence of the PCR reaction buffer from Promega kit containing 0.1 nmol of oligonucleotide (SEQ ID NO. 2) and 0.1 mmol of oligonucleotide (SEQ ID NO. 3), 10 pmol each of nucleotides dATP, dCTP, dGTP, and dTTP, 62.5 pmol of MgCl₂ (Promega) and 5 μl of the reverse transcription reaction medium obtained previously. Amplification conditions were as follow: Denaturating at 94° C. for 1 minute, anneling at 55° C. for 2 minutes, and elongating at 72° C. for 1 minute, each for 35 cycles. The PCR product was analysed by electrophoresis on a 20 g/l agarose gel to screen for the presence of the appropriate-size band by using the fluorescent dye ethidium bromide. The PCR product was then isolated, purified by phenol-chloroform extraction, dried and resuspended in distilled water according to the method described by Sambrook et al. (25).

Cloning

The obtained purified PCR product was then subjected to the ligation reaction into the pCR® II plasmid vector of the TA Cloning kit (Invitrogen). The reagents of this kit and the reaction conditions performed according to the manufacturer's recommendations were used. The ligation product was then introduced in INVαF′ E. Coli strain by using the reagents and the transformation procedure of the TA Cloning kit (Invitrogen). The screening for inserts was performed by using blue-white color selection. The sequencing of inserts obtained was performed by using the ABI DNA sequencer. The resulting vector was termed (1) (pCR® II/SMN-cDNA).

Construction of the Expression Plasmids for Human SMN Protein

To construct the expression plasmids for human SMN protein, the following systems for expression of SMN recombinant protein were performed:

1. Insect Expression Systems

1. 1. Using the Bac-to-Bac® Baculovirus Expression System

From the vector (1) obtained previously, the BamHI to XhoI fragment containing the cDNA coding sequences of SMN protein was isolated and subjected to the ligation reaction into the baculovirus transfer vector pFastBac™ HTb (Invitrogen) predigested by BamHI and XhoI and predephosphorylated with calf intestinal alkaline phosphatase (Boerhinger Mannheim, GmbH, Germany). The reaction was conducted in the presence of the reagents for the ligation of the TA Cloning kit (Invitrogen); the reaction conditions used are according to the manufacturer's recommendations. The ligation product was then introduced in INVαF′ E. Coli strain (Invitrogen) by using the reagents and the transformation procedure of the TA Cloning kit (In vitrogen). The screening for inserts was based on the presence of white colonies. The resulting vector thus obtained was termed (2) (pFastBac™ HTb/SMN-cDNA). This vector was then introduced in the DH10Bac™ E. Coli competent cells (Invitrogen) by using the reagents and the transformation procedure of the Bac-to-Bac® Baculovirus Expression System (Invitrogen). The screening for recombinant bacmids was performed by using blue-white color selection. The verification of the presence of SMN-cDNA's insert in the recombinant bacmid was performed by PCR amplification using the M13 forward (−40) and M13 reverse primers (Invitrogen). The reaction conditions used are according to the manufacturer's recommendations. The PCR product was analysed by electrophoresis on a 20 g/l agarose gel to screen for the presence of the appropriated-size band by using the fluorescent dye ethidium bromide. The PCR product was then isolated, purified by phenol-chloroform extraction, dried and resuspended in distilled water according to the method described by Sambrook et al. (25). The sequencing of SMN-cDNA's insert was performed by using the ABI DNA sequencer. The recombinant bacmid thus obtained was termed (3).

1. 2. Using the Bac-N-Bac™ Baculovirus Expression System

From the vector (2) obtained previously, the BamHI to XhoI fragment containing the cDNA coding sequence of SMN protein was isolated and subjected to the ligation reaction into the baculovirus transfer vector pBlueBacHis2 A (Invitrogen) predigested by BamHI and XhoI and predephosphorylated with calf intestinal alkaline phosphatase (Boehringer Mannheim, GmbH, Germany). The reaction was conducted in the presence of the reagents for the ligation of the TA Cloning kit (Invitrogen); the reaction conditions used are according to the manufacturer's recommendations. The ligation product was then introduced in INVαF′ E. Coli strain (Invitrogen) by using the reagents and the transformation procedure of the TA Cloning kit (Invitrogen). The screening for inserts was performed by using blue-white color selection. The resulting vector thus obtained was termed (4) (pBlueBacHis2 A/SMN-cDNA).

2. Prokaryotic Expression Systems

From the vector (2) obtained previously, the BamHI to XhoI fragment containing the cDNA coding sequences of SMN protein was isolated and subjected to the ligation reaction into the pET-28a (+) plasmid vector (Novagen) predigested by BamHI and XhoI and predephosphorylated with calf intestinal alkaline phosphatase (Boehringer Mannheim, GmbH, Germany). The reaction was conducted in the presence of the reagents for the ligation of the TA Cloning kit (Invitrogen); the reaction conditions used are according to the manufacturer's recommendations. The ligation product was then introduced in INVαF′ E. Coli strain (Invitrogen) by using the reagents and the transformation procedure of the TA Cloning kit (Invitrogen). The screening for inserts was based on the presence of white colonies. The resulting vector thus obtained was termed (5) (pET-28a (+)/SMN-cDNA).

### V. RESULTS AND DISCUSSION

Cloning of Human SMN Gene

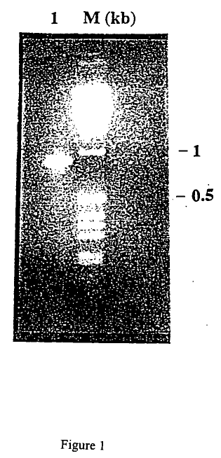

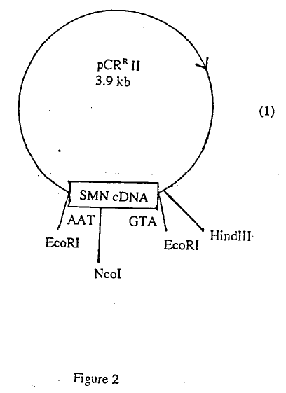

In order to investigate the molecular mechanism of SMA, intense efforts have been directed toward the identification of SMN-cDNA clones which encode the amino acid sequences of human SMN (14). The availability of the SMN-cDNA clones has permitted the construction of the expression plasmid pEGFP-C3/SMN for human SMN recombinant protein (28). Up to now, the only approach to identify SMN-cDNA clones from the cDNA library is by using the synthesized oligonucleotide probes (14); however, this approach is costly, time-consuming (requiring labor intensive steps) and it requires highly skilled personnel. Furthermore, not many laboratories have the equipment or capability to perform such a procedure. In contrast, the present invention is the development of a procedure that is easy, simple and cost-effective which can be performed in any laboratory; this procedure starts with the human SMN-mRNA and includes the utilization of the RT and PCR reactions in order to obtain SMN-cDNA for cloning. As shown in FIG. 1, the PCR product of ˜0.9 kb of the human SMN gene was successfully amplified by two synthesized oligonucleotides (SEQ ID NO. 2) and (SEQ ID NO. 3). The obtained PCR product was subcloned into the pCR® II plasmid vector of 3.9 kb (Invitrogen). The analysis of the sequence of inserts showed that the DNA sequences of the PCR product completely matched with the sequences of SMN-mRNA described by Lefebvre et al. (14). Then, the orientation of SMN-cDNA's insert in the plasmid vector (1) (pCR® II/SMN-cDNA) (FIG. 2) was determined by the digestion of (1) with NcoI and HindIII; the result shows two fragments of 4,220 and 565 bp (data not shown).

Construction of the Expression Plasmids for Human SMN Protein

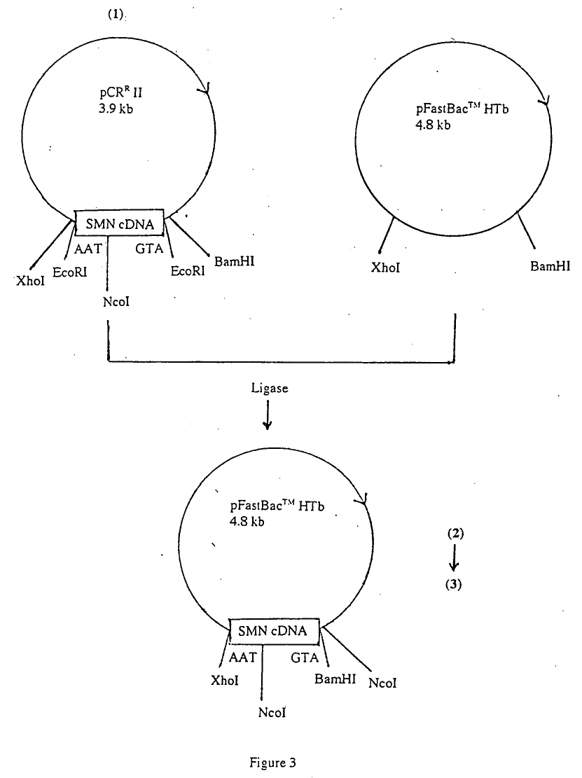

A schematic representation of the different cloning steps for constructing the expression plasmids for human SMN recombinant protein are given in FIGS. 3-5.

1. Insect Expression Systems

Insect expression systems are relative newcomers to the gene expression arena. As higher eukaryotes, insects cells offer many mammalian posttranslational modifications. Insect cells have proven to be an excellent host for recombinant protein expression. They are often chosen for protein production because they are a higher eukaryotic with easy cell culture and can be readily adapted to high-density suspension culture for large-scale expression.

1.1 Using the Bac-to-Bac® Baculovirus Expression System

The Bac-to-Bac® Baculovirus Expression system is the fastest route available for producing recombinant baculovirus. This system uses a unique bacmid shuttle vector that combines with an expression cassette by siote-specific transposition to create an expression bacmid. The expression bacmid is then transfected into insect cells to generate high-titer, ready-to-use recombinant baculovirus. With Bac-to-Bac®, recombinant protein can be expressed in as little as nine days. The Bac-to-Bac® HT baculovirus expression system (Invitrogen) is used. This system offers the components necessary for expression and purification of histidine tagged recombinant proteins in Sf9, Sf21, or High Five™ cells. The system includes the pFastBac™ HTb vector of 4.8 kb, the DH10Bac™ E. Coli competent cells for cloning. Expression of the recombinant fusion proteins from the pFastBac™ HTb vector is driven by the polyhedrin promotor. Proteins expressed are fused at the N-terminus to a tag of six tandem histidine residues and a TEV protease cleavage site for removal of histidine tag following protein purification. The histidine residues create a high-affinity metal binding site to allow purification of recombinant fusion proteins on nicken-chelating resin. In the present work, a schematic representation of the different cloning steps for constructing the expression plasmid for SMN protein using the pFastBac™ HTb vector is given in FIG. 3. The correct orientation for SMN protein expression of the SMN-cDNA's insert in the plasmid vector (2) was confirmed by the digestion of (2) with NcoI which gave two fragments of 5,120 and 565 bp (data not shown). The verification of the presence of SMN-cDNA's insert in the recombinant bacmid (3) using PCR technique (in the presence of M13 forward (−40) and M13 reverse primers) gave a DNA fragment of 3,315 bp (data not shown). The DNA sequences of the obtained PCR product completely matched with the sequences of SMN-mRNA described by Lefebvre et al. (14).

1.2. Using the Bac-N-Bac™ Baculovirus Expression System

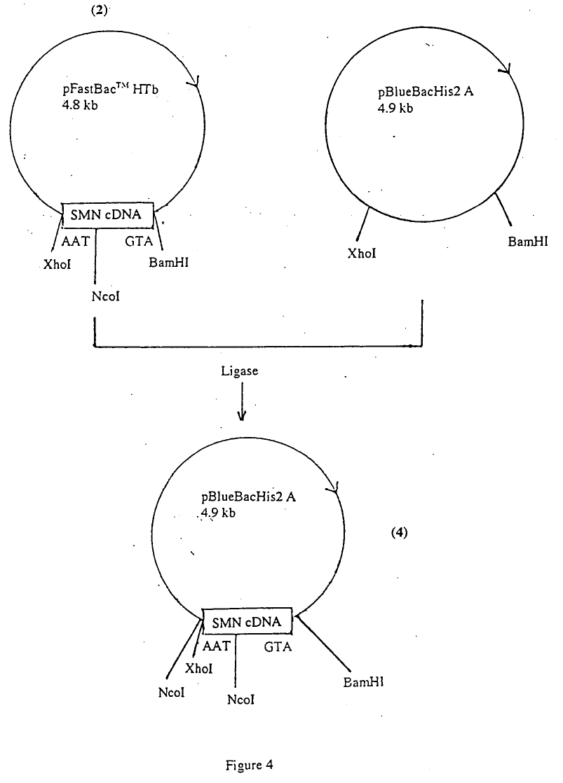

The Bac-N-Bac™ Baculovirus Expression system has been used for over a decade to produce high levels of recombinant proteins. The baculovirus transfer vector pBlueBacHis2 A (Invitrogen) is used. The pBlueBacHis2 A is a 4.9 kb polyhedrin promotor-based vector that is designed to create N-terminal fusion proteins produced in the baculovirus system. The vector's small size allows an easier cloning of the gene of interest. Expression of β-galactosidase following recombination with Bac-N-Blue™ Linear DNA promotes simplified screening of recombinant plaques. Proteins expressed from pBlueBacHis2 A are fused at the N-terminus to a tag of six tandem histidine residue and an enterokinase cleavage site (the Xpress™ tag). The histidine residues create a high-affinity metal binding site to allow purification of recombinant fusion proteins on nickel-chelating resin. The Xpress™ tag is easily cleaved away from the protein using enterokinase. Expression and purification of the Xpress™ fusion protein is easily tracked using the Anti-Xpress™ Antibody which recognizes an epitope located in the Xpress™ tag. In the present work, a schematic representation of the different cloning steps for constructing the expression plasmid for SMN protein using the pBlueBacHis2 A vector is given in FIG. 4. The correct orientation for SMN expression of the SMN-cDNA's insert in the plasmid vector (4) was confirmed by the digestion of (4) with NcoI which gave two fragments of 5,465 and 320 bp (data not shown).

2. Prokaryotic Expression Systems

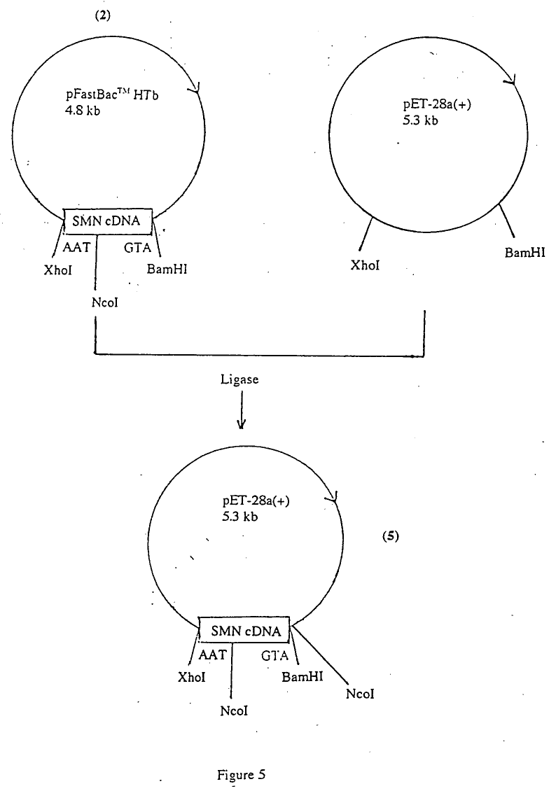

Prokaryotic hosts, especially E. Coli, have been widely used for the expression of recombinant proteins. The Prokaryotic Expression systems offers several advantages as recombinant protein expression hosts, including easy manipulation, rapid growth and simple media requirements. Prokaryotic expression systems are well suited for expression of proteins that will be used in antibody production and for structural studies. The Prokaryotic Expression systems use the T7 expression system which allows high-level expression from the strong bacteriophage T7 promoter and T7 RNA polymerase. The T7 expression system is ideal for expressing soluble non-toxic recombinant protein in E. Coli. In the present work, the pET-28a (+) vector (Novagen) is used; this vector of 5.3 kb carry an N-terminal His Tag/thrombin/T7 Tag configuration plus an optional C-terminal His Tag sequence. In the present work, a schematic representation of the different cloning steps for constructing the expression plasmid for SMN protein using pET-28a (+) vector is given in FIG. 5. The correct orientation for SMN expression protein of the SMN-cDNA's insert in the plasmid vector (5) was confirmed by the digestion of (5) with NcoI which gave two fragments of 5,620 and 565 bp (data not shown).

### VI. CONCLUSION

The present invention is a new approach to the cloning of human SMN gene based on the RT and PCR reactions. In comparison to the old way of isolating SMN-cDNA clones from the cDNA library by using synthesized oligonucleotide probes, the procedure for cloning of human SMN gene via RT-PCR reactions developed herein is cost-effective, not time-consuming, and is suited for any laboratory. As a result, the construction of the expression plasmids for human SMN protein can be performed by any laboratory. The present invention makes it easier to obtain full-length SMN protein which is valuable for biochemical and biological analyses that may elucidate the molecular mechanism of SMA. Knowing the molecular mechanism of SMA will allow the exploration of gene therapy in SMA.

### VIII. REFERENCES

1. Roberts, D. F.; Chavez, J.; Court, S. D. M. The genetic component in child mortality. Arch. Dis. Child 45, (1970) 33-38.
2. Pearn, J. The gene frequency of acute Werdnig-Hoffmann disease (SMA type I). A total population survey in North East England. J. Med. Genet. 10, (1973) 260-265.
3. Pearn, J. Incidence, prevalence, and gene frequency studies of chronic childhood spinal muscular atrophy. J. Med. Genet. 15, (1978) 409-413.
4. Czeizel, A.; Hamula, J. A Hungarian study on Werdnig-Hoffmann disease. J. Med. Genet. 26, (1989) 761-763.
5. Munsat, T. L. Workshop report: International SMA collaboration. Neuromusc. Disord. 1, (1991) 81.
6. Werdnig, G. Die fruhinfantile progressive spinal atrophie. Arch. Psych. 26, (1894) 706-744.
7. Hoffmann, J. Uber die hereditare progressive spinal muskelatrophie im Kindesalter. Munchen Med. Wschr. 47, (1900) 1649-1651.
8. Kugelberg, E.; Welander, L. Heredo-familial juvenile muscular atrophy simulating muscular dystrophy. Arch. Neurol. Psych. 75, (1956) 500-509.
9. Brzustowicz, L. M.; Lehner, T.; Castilla, L. H.; et al. Genetic mapping of chronic childhood-onset spinal muscular atrophy to chromosome 5q 11.2-q 13.3. Nature 344, (1990) 540-541.
10. Gilliam, T. C.; Brzustowicz, L. M.; Castilla, L. H.; et al. Genetic homogeneity between acute and chronic forms of spinal muscular atrophy. Nature 345, (1990) 823-825.
11. Melki, J.; Abdelhak, S.; Sheth, P.; et al. Gene for proximal spinal muscular atrophies maps to chromosome 5q. Nature 344, (1990) 767-768.
12. Melki, J.; Sheth, P.; Abdelhak, S.; et al. Mapping of acute (type I) spinal muscular atrophy to chromosome 5q12-q14. Lancet 336, (1990) 271-273.
13. Melki, J.; Lefebvre, S.; Burglen, L.; et al. De novo and inherited deletions of the 5q13 region in spinal muscular atrophies. Science 264, (1994) 1474-1477.
14. Lefebvre, S.; Burglen, L.; Reboullet, S.; et al. Identification and characterization of a spinal muscular atrophy-determining gene. Cell 80, (1995) 155-165.
15. Roy, N.; Mahadevan, M.; Mclean, M.; et al. The gene for neuronal apoptosis inhibitory protein is partially deleted in individuals with spinal muscular atrophy. Cell 80, (1995) 167-178.
16. Fischer, U.; Liu, Q.; Dreyfuss, G. The SMN-SIP-1 complex has an essential role in spliceosomal snRNP biosynthesis. Cell 90, (1997) 1023-1029.
17. Liu, Q.; Fischer, U.; Wang, F.; Dreyfuss, G. The spinal muscular atrophy disease gene product, SMN, and its associated protein SIP-I are in complex with spiceosomal snRNP proteins. Cell 90, (1997) 1013-1021.
18. Pellizzoni, L.; Kataoka, N.; Charroux, B.; Dreyfuss, G. A novel function for SMN, the spinal muscular atrophy disease gene product, in pre-mRNA splicing. Cell 95, (1998) 615-624.
19. Schrank, B.; Gotz, R.; Gunneron, J.; Ure, J.; Toyka, K.; Smith, A.; Sendtner, M. Inactivation of the survival motor neuron gene, a candidate gene for human spinal muscular atrophy, leads to massive cell death in early mouse embryos. Proc. Natl. Acad. Sci. USA 94, (1997) 9920-9925.
20. Burlet, P.; Huber, C.; Bertrandy, S.; Ludosky, M. A.; Zwaenepoel, I.; Clermont, O.; Roume, J.; Delezoide, A. L.; Cartaud, J.; Munnich, A.; Lefebvre, S. The distribution of SMN protein complex in human fetal tissues and its alteration in spinal muscular atrophy. Hum. Mol. Genet. 7, (1998) 1927-1933.
21. Lefebvre, S.; Burlet, P.; Liu, Q.; Bertrandy, S.; Clermont, O.; Munnich, A.; Dreyfuss, G.; Melki, J. Correlation between severity and SMN protein level in spinal muscular atrophy. Nature Genet. 16, (1997) 66-70.
22. Monani, U. R.; Sendtner, M.; Coovert, D. D.; Parsons, D. W.; Andreassi, C.; Le, T. T.; Jablonka, S.; Schrank, B.; Rossoll, W.; Prior, T. W.; Morris, G. E.; Burghes, A. H. M. The human centromeric survival motor neuron gene (SMN 2) rescues embryonic lethality in Smm^{-/-} mice and results in a mouse with spinal muscular atrophy. Hum. Mol. Genet. 9, (2000) 333-339.
23. Jablonka, S.; Schrank, B.; Kralewski, M.; Rossoll, W.; Sendtner, M. Reduced survival motor neuron (Smn) gene dose in mice leads to motor neuron degeneration: an animal model for spinal muscular atrophy type III. Hum. Mol. Genet. 9, (2000) 341-346.
24. Li, H. M. H.; Chang, J. G.; Jong, Y. J.; Wu, M. H.; Wang. N. M.; Tsai, C. H.; Li, H. A mouse model for spinal muscular atrophy. Nature Genet. 24, (2000) 66-70.
25. Sambrook, J.; Fritsch, E. F.; Maniatis, T. Extraction, purification and analysis of messenger RNA from eukaryotic cells. In Molecular Cloning, a Laboratory Manual, 2^{nd} Ed. Cold Spring Harbor Laboratory Press: Cold Spring Harbor, N.Y. (1989) 7.28-7.52.
26. Saiki, R. K.; Scharf, S.; Faloona, F; Mullis, K. B.; Horn, C. T.; Erlich, H. A.; Arnheim, N. Amplification of β-globine genomic sequences and restriction site and analysis for diagnosis of sickle cell anemia. Science 230 (1985) 1350-1354.
27. Kawasaki, E. S.; Wang, A. M. Detection of gene expression. In PCR Technology, Erlich, H. A., Ed. Stockton: New York (1989) 89-97.
28. Arnold, A. S.; Gueye, M.; Ronde, P.; Warter, J. M.; Poindron, P.; Gies, J. P. Construction of a plasmid containing human SMN, the SMA determining gene, coupled to EGFP. Plasmid 47, (2002) 79-87.

What is claimed is:

1. The procedure for cloning human SMN gene based on the reverse transcription (RT) and the polymerase chain reaction (PCR) using the synthesized oligonucleotides (SEQ ID NO. 1) for RT, and (SEQ ID NO. 2) and (SEQ ID NO. 3) respectively for PCR, comprising:
Isolating RNA;
Performing RT reaction using the synthesized oligonucleotide
5′ TGGCAGACTTAC 3′ (SEQ ID NO. 1) under the following conditions: 90° C. for 2 minutes; 0° C. for 1 minute; 25° C. for 10 minutes; 42° C. for 45 minutes;
Performing PCR reaction using the synthesized oligonucleotides
5′ ATGGCGATGAGCAGCGG 3′ (SEQ ID NO. 2) and
5′ TTAATTTAAGGAATGTGAGCAC 3′ (SEQ ID NO. 3) under the following conditions: Denaturating at 94° C. for 1 minute; annealing at 55° C. for 2 minutes; elongating at 72° C. for 1 minute each cycle, for 35 cycles.

2. The procedure for the construction of expression plasmids using the pFastBac™ HTb and the pBlueBacHis2 A transfer vectors for the purpose of obtaining human SMN protein in insect cells, comprising:
2.1. Using the pFastBac™ HTb vector:
Digesting the pFastBac™ HTh vector with BamHI and XhoI followed by dephosphorylation with calf intestinal alkaline phosphatase;
Digesting the vector (1) pCR® II/SMN-cDNA with BamHI and XhoI and isolating the resulting fragment containing the cDNA coding sequences of SMN protein, SMN-cDNA;
Ligating the SMN-cDNA fragment to the pFastBac™ HTb vector and introducing the ligation product in INVα F′ E. Coli strain;
Screening for inserts based on the presence of white colonies, as a result of which the vector (2) pFastBac™ HTb/SMN-cDNA is selected;
Introducing the vector (2) in DH10Bac™ E. Coli competent cells;
Screening for recombinant bacmids in DH10Bac™ E. Coli using blue-white color selection, then verifying the presence of SMN-cDNA's insert in the recombinant bacmids by PCR amplification using the M13 forward (−40) and M13 reverse primers, as a result of which the recombinant bacmid (3) is selected;
2.2. Using the pBlueBacHis2 A vector:
Digesting the pBlueBacHis2 A vector with BamHI and XhoI followed by dephosphorylation with calf intestinal alkaline phosphatase;
Digesting the vector (2) pFastBac™ HTb/SMN-cDNA with BamHI and XhoI and isolating the resulting fragment containing the cDNA coding sequences of SMN protein, SMN-cDNA;
Ligating the SMN-cDNA fragment to the pBlueBacHis2 A vector and introducing the ligation product in INVα F′ E. Coli strain;
Screening for inserts using blue-white color selection, as a result of which the vector (4) pBlueBacHis2 A/SMN-cDNA is selected.

3. The procedure for the construction of expression plasmids using the pET-28a (+) transfer vector for the purpose of obtaining human SMN protein in bacteria, comprising:
Digesting the pET-28a (+) vector with BamHI and XhoI followed by dephosphorylation with calf intestinal alkaline phosphatase;
Digesting the vector (2) pFastBac™ HTb/SMN-cDNA with BamHI and XhoI and isolating the resulting fragment containing the cDNA coding sequences of SMN protein, SMN-cDNA;
Ligating the SMN-cDNA fragment to the pET-28a (+) vector and introducing the ligation product in INVα F′ E. Coli strain;
Screening for inserts based on the presence of white colonies, as a result of which the vector (5) pET-28a (+)/SMN-cDNA is selected.


SEQUENCE LISTING (3 sequences)

SEQ ID NO: 1 - DNA, 12 bp, Human
tggcagacttac

SEQ ID NO: 2 - DNA, 17 bp, Human
atggcgatgagcagcgg

SEQ ID NO: 3 - DNA, 22 bp, Human
ttaatttaaggaatgtgagcac